(19) United States
(12) Patent Application Publication
(10) Pub. No.: US 2026/0146378 A1
(43) Pub. Date: May 28, 2026

(54) HIGH CROSS-DIRECTIONAL ELONGATION NONWOVEN FABRICS

(71) Applicant: FIBERTEX PERSONAL CARE A/S, Aalborg Ost (DK)

(72) Inventors: Lasse Kristoffer TAIDAL, Aalborg (DK); Brian UDENGAARD, Lystrup (DK)

(73) Assignee: FIBERTEX PERSONAL CARE A/S, Aalborg Ost (DK)

(21) Appl. No.: 19/454,473

(22) Filed: Jan. 21, 2026

Related U.S. Application Data
(63) Continuation of application No. PCT/EP2024/073884, filed on Aug. 27, 2024.

(30) Foreign Application Priority Data
Aug. 28, 2023 (EP) 23193634.5

(51) Int. Cl. D04H 1/50; D04H 1/4291; D04H 1/558; D04H 1/56

(52) U.S. Cl. CPC D04H 1/50; D04H 1/4291; D04H 1/558; D04H 1/565; D10B 2321/02; D10B 2509/026

(57) ABSTRACT

The invention relates to nonwoven sheets having high inherent cross-machine directional elongation, the sheets comprising two facing layers of spunbonded nonwoven material and a middle layer of meltblown nonwoven material, wherein the spunbonded material of the facing layers comprises crimped fibers, wherein the fibers of the meltblown material of the middle layer comprise a thermoplastic elastomer, and wherein the nonwoven sheet is underbonded. The invention also relates to a method of making such nonwovens, laminates comprising such nonwoven and an elastically stretchable material, and hygiene articles comprising such nonwoven sheets or laminates.

This application is a continuation of PCT/EP2024/073884, filed Aug. 27, 2024, which claims priority to European Patent Application No. 23193634.5, filed Aug. 28, 2023, both of which are incorporated in their entirety by reference herein.

The invention relates to stretchable nonwoven sheets comprising two spunbonded facing layers and a meltblown middle layer, and a method of making these sheets.

The hygiene industry uses nonwoven sheets on a large scale as materials in baby diapers and adult incontinence products. In many instances, for example to make back-ears in open diapers, the materials are required to be elastically stretchable. One option to make elastically stretchable materials is laminating stretchable nonwovens and elastic films or strands. The elastic film imparts the elastic properties, while the nonwoven sheets layers make the material look and feel as an integrated part of the diaper. In such configuration, while the nonwoven sheets do not need to have elastic properties themselves, their elongation, meaning their ability to stretch, is the limiting factor for overall elongation of the back ear. Hence, the nonwoven material should have significant elongation properties. More specifically, as in production methods of open diapers, the length direction of the back ears requiring elastic stretch is usually the cross-machine direction (CD) of the nonwoven sheets, for nonwoven sheets to be laminated with an elastic film or strand for making diaper back ears, it is advantageous to exhibit significant elongation specifically in cross-machine direction.

In the state of the art, nonwoven sheets of satisfactory inherent CD direction elongation that can be produced at reasonable cost are not available. As a workaround, it is known to pre-stretch the elastic film and apply the nonwoven sheet to both sides of the elastic laminate while it is stretched. When the elastic film is allowed to contract, the nonwoven sheets contract and drape. Downsides of this approach are an overuse of nonwovens and a higher thickness and poor visual impression of the final product. Another known method is to pre-stretch the nonwoven sheets by ring-rolling or similar methods, which leads to partly breaking the nonwoven structure and enhanced elongation properties. However, the activation leads to an additional step in production and the partly broken structure of the nonwoven creates a less desired visual impression.

An object of this invention is to provide a nonwoven sheet that has satisfactory inherent CD elongation and can be produced at reasonable cost.

Against this background, the present invention proposes a method for making a nonwoven sheet, the nonwoven sheet comprising two facing layers of spunbonded nonwoven material and a middle layer of meltblown nonwoven material, wherein the spunbonded material of the facing layers comprises crimped fibers, the method comprising the following in-line steps: spinning crimped multicomponent fibers and laying them onto a moving spinbelt to form a web preceding one of the facing layers; meltblowing fibers and laying them onto the surface of the web formed in the previous step to form a web preceding the middle layer; spinning crimped multicomponent fibers and laying them onto the surface of the web formed in the previous step to form a web preceding the other one of the facing layers; and bonding the adjacent webs to form the nonwoven sheet by point-bond hot calendering. According to the invention, the fibers of the meltblown material of the middle layer comprise a thermoplastic elastomer and the temperature of the engraved roll of the calender is at least 10° C. and/or 5% lower than the melting temperature of the lowest melting component of the multicomponent fibers.

The temperature of the calender rolls refers to the surface temperature of the calender rolls. In practice, the temperature is often not measured on the surface, but set by setting the temperature of a heating oil used to heat the rolls, usually by being passed through conduits inside the rolls. The relation between the oil temperature and the actual surface temperature is not linear and influenced by multiple factors. In any case, however, the surface temperature is never higher than the oil temperature. In other words, the definition that the (surface) temperature of the engraved roll of the calender is at least 10° C. and/or 5% lower than the melting temperature of the lowest melting component of the multicomponent fibers encompasses a (more narrow) definition the temperature of a heating oil used to heat the rolls is at least 10° C. and/or 5% lower than the melting temperature of the lowest melting component of the multicomponent fibers.

The present invention further proposes a nonwoven sheet made by a method of the invention, the nonwoven sheet comprising two facing layers of spunbonded nonwoven material and a middle layer of meltblown nonwoven material, wherein the spunbonded material of the facing layers comprises crimped fibers, wherein the meltblown material of the middle layer comprises a thermoplastic elastomer, and wherein the nonwoven sheet is underbonded.

Point-bond hot calendering involves the use of a two-roll nip consisting, in most cases, of a heated engraved roll (e-roll) and a typically also heated smooth roll (s-roll). The multilayer web is fed by an apron leading to a calender nip and the fiber temperature is locally raised to the point at which tackiness and melting cause fiber segments caught between the tips of engraved points and the smooth roll to adhere together.

The process temperature and, in particular, the temperature of the engraved calender roll is a significant parameter for bonding quality. For a given nip line pressure, e.g. 20 to 100 N/mm, preferably 30 to 70 N/mm, and line speed, e.g. 100 to 400 m/min, preferably 200 to 350 m/min, the breaking strength reaches a maximum at a critical bonding temperature. When temperature of the engraved roll of the calender is significantly lower than the melting temperature of the lowest melting component of the multicomponent fibers, it will be significantly below the critical bonding temperature, and the sheet, specifically the spunbonded nonwoven material will become underbonded. This means that the quantity of adhered fiber segments at the engraved points is significantly reduced when compared to a critical bonding temperature.

In the nonwoven sheet made according to the invention, the meltblown middle layer acts as an adhesive to the spunbonded facing layers. Spunbonded materials comprising crimped fibers have a certain inherent elongation. The inherent elongation of even these materials, however, is still limited. Inherent elongation can be increased by underbonding. However, when underbonding sheets merely consisting of one or more layers of spunbonded material, the sheets tend to be unstable and fuzzy. The present invention uses a meltblown layer of thermoplastic elastomer material between two facing layers of spunbonded material. The meltblown layer acts as an adhesive between the facing layers due to an inherent stickiness of the freshly formed thermoplastic elastomer meltblown fibers. It was found that the stickiness of the elastic meltblown layer enables for underbonding the facing layers without detriment to fabric stability. At the same time, as the meltblown fibers are made from a thermoplastic elastomer and the layer is hence stretchable in itself, the additional layer does not hinder elongation, at least to an extent that would cancel out the positive effects of the ability to underbond.

In contrast to technologies using spunbonded layers formed from thermoplastic elastomer materials, with an aim to provide nonwoven materials that are inherently elastically stretchable and do not need to be laminated with elastic films or strands, the sheets of the present invention are very stretchable, but have no or only little inherent elasticity. In other words, when stretching the sheets of the invention, at least part of the deformation will be permanent. The meltblown layer, while formed from a thermoplastic elastomer, only functions as a connecting element for structural integrity of an underbonded web, but does not have sufficient strength to impart inherent elastic properties to the sheet.

If reference is made herein to the temperature of the engraved roll of the calender being at least a certain percentage, e.g. 5% lower than the melting temperature of the lowest melting component of the multicomponent fibers, reference is made to the melting temperature as expressed in ° C. Hence, to give an example, for a polymer component of a 140° C. melting point, 5% would be 7° C., and at least 5% lower than 140° C., in absolute numbers, would be 133° C. or lower.

In embodiments, the temperature of the engraved roll of the calender can at least 20° C., or at least 30° C., or at least 40° C. lower than the melting temperature of the lowest melting component of the multicomponent fibers. In embodiments, the temperature of the engraved roll of the calender is least 10%, or at least 20%, or at least 30% lower than the melting temperature of the lowest melting component of the crimped multicomponent fibers. In embodiments, the absolute temperature of the engraved roll of the calender can be 130° C. or less, 120° C. or less, 110° C. or less, 110° C. or less, or even 90° C. or less. As explained further above, the temperature refers to the surface temperature of the roll in principle, but the feature encompasses a (more narrow) definition the temperature of a heating oil used to heat the rolls is at least a certain absolute temperature (C) or relative temperature (%) lower than the melting temperature of the lowest melting component of the multicomponent fibers, or is a certain temperature (° C.) or less.

In embodiments, the temperature of the smooth roll of the calender is smaller than the temperature of the engraved roll of the calender. Preferably, the temperature difference between the rolls is less than 20° C. Also here, the definition, in embodiments, can be applicable to both a comparison of the surface temperatures of the rolls and the temperature of heating oil used to heat the rolls.

The fibers forming for the facing layers can comprise crimped multicomponent fibers or consist of crimped multicomponent fibers. For sufficient elongation, it is preferred that at least 70 wt. %, preferably at least 80 wt. %, more preferably at least 90 wt. % of the fibers are crimped multicomponent fibers. The crimped multicomponent fibers make these layers high loft with improved softness, in addition to an improved flexibility and elongation.

The crimped multicomponent fibers are of any asymmetric cross-sectional distribution and are preferably bicomponent fibers. Preferred are side-by-side fibers, but they can also be of eccentric-sheath-core or other known configuration. In a preferred embodiment, one of the components of the fibers is polypropylene (PP) and another component of the fibers is a propylene-α-olefin copolymer material (co-PP). The copolymer is preferably a poly(propylene-ethylene) copolymer. In another embodiment, the components are both polypropylene, with the polypropylene differing from each other in properties such that a crimp in the fibers results.

The melting temperatures of polypropylene (PP) materials as used in the facing layers can, in one embodiment, range from 150-170° C., preferably 155-165° C. The melting temperatures of propylene-α-olefin copolymer material (co-PP) materials as used in the facing layers can, in one embodiment, range from 140-160° C., preferably 145-155° C. Melting temperatures (Tₘ) as given herein are generally to be understood as having been determined with DSC according to ISO 11357-3.

Typical values for melt flow rates of the polymers used in the bicomponent fibers can be between 10 and 50 g/10 min. Melt flow rates as indicated herein are generally to be understood as being determined according to ISO 1133 with conditions being 230° C. and 2.16 kg.

The weight ratio of the one component to the other component preferably lies between 20/80 and 80/20, more preferably between 30/70 and 70/30, and yet more preferably between 40/60 and

Each spunbonded facing layer can comprise one single layer or can comprise two or even more layers in an SS configuration.

For sufficient stickiness, it is preferred that at least 10 wt. %, preferably at least 50 wt. %, more preferably at least 90 wt. % of the material forming for the meltblown fibers of the middle layer is a thermoplastic elastomer. In an embodiment, the meltblown fibers consist of 100 wt. % thermoplastic elastomer. From a viewpoint of stickiness, 100 wt. % of TPE can be preferred. For processability, however, mixing with another thermoplastic polymer like a regular thermoplastic polypropylene can be of advantage. When another thermoplastic polymer is used in conjunction with the TPE material for making the meltblown fibers, it is preferred that this material has a melt flow rate. The melt flow rates of the polymer(s) used for meltblowing are typically higher than that of the polymers used for spunbonding. Typical values exceed 500 g/10 min determined according to ISO 1133 with conditions being 230° C. and 2.16 kg.

The thermoplastic elastomer can be a thermoplastic polyolefin elastomer (TPE-o), preferably a thermoplastic polyolefin elastomer comprising propylene-α-olefin copolymers. Suitable TPE-o materials for use in the context of the present invention are comprised, for example, in the Vistamaxx™ series from ExxonMobil. An example comprises Vistamaxx™ EXP520. Alternatively or additionally, meaning as a mixture, other thermoplastic elastomer materials like especially thermoplastic polyurethanes (TPU) or styrenic block copolymers (TPE-s) may be used. In one embodiment, up to 20 wt.-% and preferably up to 10 wt.-% of a thermoplastic olefin, such as a homo-polypropylene may be contained in the thermoplastic elastomer material next to the thermoplastic elastomer.

The meltblown middle layer can comprise one single layer or can comprise two or even more layers in an MM configuration.

The nonwoven sheet according to the invention preferably has a tensile strength at @100% elongation of less than 0.65 and preferably less than 0.55 of its tensile strength @break, with tensile strength at @100% elongation and tensile strength @break both being determined according to WSP 110.4. Preferred quotients of tensile @100% to tensile @break are less than 0.50, less than 0.45 or even less than 0.40.

Further, the nonwoven sheet according to the invention preferably has an extension at break in cross-machine direction (CD) of at least 150%, when measured according to WSP 110.4. CD elongation at 5N values are preferably at least 75%, when measured according to WSP 110.4.

In one embodiment, the basis weight of each facing layer is 5-40 g/m², preferably between 10-25 g/m². The basis weight of the meltblown middle layer may, in one embodiment, be 10 g/m² or less.

The number of bonding points per cm² of fabric surface may be lower than 100 and preferably lower than 70, and on the other hand preferably higher than 30. The total area of the fabric surface taken up by the areal bonding points in one embodiment is less than 18% and preferably less than 15%, and on the other hand preferably higher than 8%.

The facing layers on either side of the middle layer may be identical or different. They can individually be configured according to embodiments as described above.

The invention further relates to a laminate comprising a nonwoven sheet according to the invention and an elastically stretchable material. Elastically stretchable materials can be elastic films or strands, elastomeric coatings, elastomeric nonwovens, scrims, or the like. Preferably, the elastically stretchable material is sandwiched between two nonwoven sheets according to the invention. Lamination can be done by ultrasonic bonding, thermal bonding, gluing, and other known techniques. When laminating, the film can in one embodiment be pre-stretched and then relaxed, so the nonwoven sheets contract and drape. While this may be considered a less than optimal solution, as compared to the prior art that uses such workaround, the higher inherent elongation of the nonwoven sheets of the invention allow for reduction in the degree of pre-stretching and hence thickness increase and material use. Also, while this may be considered a less than optimal solution, the nonwoven sheets of the invention before lamination or the laminates of the invention can be subjected to ring-rolling for pre-stretch, however, with less degree of pre-stretch and hence impact than with less stretchable nonwoven sheets of the prior art.

As an alternative, the nonwoven sheet according to the invention can also be used as a standalone product in a hygiene article where stretch-ability is needed.

Yet further, the invention relates to a hygiene article comprising a nonwoven sheet or laminate according to the invention. For example, the hygiene article can be an open diaper having diaper ears, and the nonwoven sheet or laminate according to the invention can form at least part of the diaper ears.

Further details and advantages of the invention will become apparent from the figures and examples described in the following. The figures show:

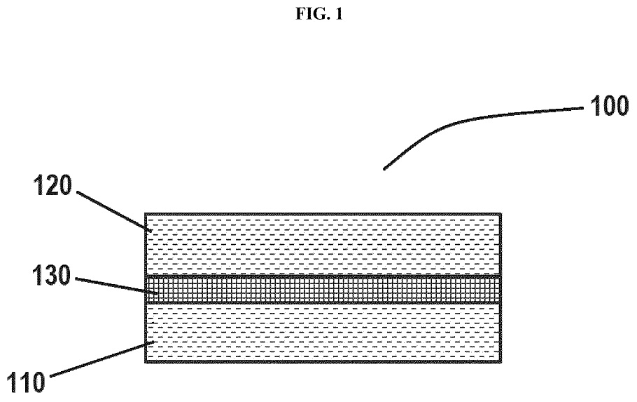

FIG. 1: a schematic cross-section of a nonwoven sheet of the invention; and

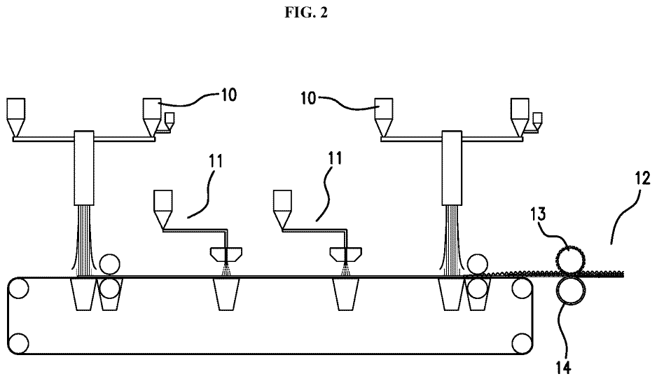

FIG. 2: an exemplary process line for manufacturing nonwoven sheets of the invention.

In FIG. 1 a schematic cross-section of a sandwich-type stretchable nonwoven sheet is shown. The sheet, generally designated with reference numeral 100, comprises a middle layer 130 that is covered with a facing layer 110 and 120, respectively, on either side. Both facing layers 110 and 120 are spunbonded nonwoven fabrics formed from crimped multicomponent fibers. The middle layer 130 is a meltblown nonwoven fabric formed from fibers comprising thermoplastic elastomer material. The layers 110, 120 and 130 are hot calender point-bonded to each other, but the fabric is underbonded due to using lower calender temperatures than temperatures that would usually be considered appropriate for bonding.

An exemplary process line for carrying out a method of the invention and manufacturing a nonwoven fabric sheet 100 as illustrated in FIG. 1 is shown in FIG. 2.

The process line comprises a conveyor belt that runs along a first spunbonding machine 10 for forming one of the facing layers 110, two meltblowing machines 11 for forming the middle layer 130, spunbonding machine 10 for forming the other of the facing layer 120, a pair of pre-compaction rollers behind every spunbonding machine 10, and downstream of the four machines 10, 11 a bonding station 12 comprising a pair of calender rolls, namely e-roll 13 and s-roll 14.

The spunbonding machines 10 each comprise two reservoirs for the polymer raw materials that form for the two components of the bicomponent fiber, a mixing and feeding channel, an extrusion die, an air channel for quenching and stretching and an air suctioning device below the conveyor belt. The meltblowing machines 11 each comprise a reservoir for the thermoplastic elastomer material, a feeder, an air manifold, a die and an air suctioning device below the conveyor belt. Pre-consolidation leads to a light fiber to fiber integration necessary to withstand further processing.

### Examples 1-7

The examples as described in Table 1 were prepared on an SMMS (spunbonded, meltblown, meltblown, spunbonded) line as shown in FIG. 2, with the spunbonded layers being loft (L, comprising bicomponent fibers). Line parameters were a line speed of 268 m/min and a nip pressure of 52 N/mm. The bond pattern was an open dot bond pattern with a bonding area of 13.6%.

TABLE 1

| | | coPP | PP/coPP | BW S1 | | coPP | PP/coPP | BW S2 |
|---|---|---|---|---|---|---|---|---|
| Ex. | PP S1 | S1 | ratio S1 | (g/m²) | PP S2 | S2 | ratio S2 | (g/m²) |
| 1 | 511A | 261S | 40/60 | 9.6 | 511A | 261S | 40/60 | 8.6 |
| 2 | 511A | 261S | 40/60 | 9.6 | 511A | 261S | 40/60 | 8.6 |
| 3 | 511A | 261S | 50/50 | 10.9 | 511A | 261S | 50/50 | 8.6 |
| 4 | 511A | 261S | 50/50 | 10.9 | 511A | 261S | 50/50 | 8.6 |
| 5 | 511A | 261S | 40/60 | 9.6 | 511A | 261S | 40/60 | 8.6 |
| 6 | 511A | 261S | 40/60 | 9.6 | 511A | 261S | 40/60 | 8.6 |
| 7 | 511A | 261S | 50/50 | 10.9 | 511A | 261S | 50/50 | 8.6 |

| | | | | BW | | |
|---|---|---|---|---|---|---|
| | oTPE in | | wt. % oTPE | MM | Calender Temp. | Calender Temp. |
| Ex. | MM | rem. MM | in MM | (g/m²) | E-roll (° C.) | S-roll (° C.) |
| 1 | | MFI800 | 0 | 3.6 | 101 | 101 |
| 2 | EXP520 | MFI800 | 75 | 3.6 | 101 | 101 |
| 3 | EXP520 | | 100 | 3.4 | 101 | 101 |
| 4 | EXP520 | | 100 | 3.4 | 130 | 120 |
| 5 | | MFI800 | 0 | 3.6 | 151 | 149 |
| 6 | EXP520 | MFI800 | 50 | 3.6 | 101 | 101 |
| 7 | EXP520 | | 100 | 3.4 | 140 | 130 |

The calender temperatures of the E- and S-rolls refer to the temperature of the oil heating the calender. The actual surface temperature of the rolls will be a bit smaller even.

The 511A polymer is a polypropylene homopolymer from the company Sabic with a narrow polymer weight distribution (M_{w}/Mₙ is 3.8), a MFR of 25 g/10 min and a Tₘ of 161° C.

The 261S polymer is a polypropylene-ethylene random polymer from the company LyondellBasell with a narrow polymer weight distribution (M_{w}/Mₙ is 3.8), a MFR of 30 g/10 min and a Tₘ of 153° C.

The MFI800 material is a standard polypropylene homopolymer meltblown grade with an MFR of 800 g/10 min.

Vistamaxx™ EXP520 is a TPE-o material from Exxon mobile of suitable MFR.

Examples 1, 5 and 7 are comparative examples outside the scope of the present invention, as they are either not underbonded or do not comprise a TPE material in the meltblown core layer. Examples 2, 3, 4 and 6 are inventive examples.

As already outlined further above, the aim for the materials of the invention is to achieve CD-elongations of minimum 150% at break. In addition, it is desired to achieve easy elongation. This can be measured by the properties of tensile elongation at 5N, tensile force at 100% elongation and tensile force at 150% elongation. In addition, the slope of the tensile-elongation-graph should be taken into account to make sure that the nonwoven web is continuously intact through the stretch of the nonwoven.

Measurement results for the nonwoven sheets of Examples 1-7 are shown in Table 2.

TABLE 2

| | | | CD tensile | CD tensile | | |
|---|---|---|---|---|---|---|
| | | | at 100% | at 150% | Sum of | Sum of |
| | CD elong. at | CD elong. at | elong. | elong. | slope | slope |
| Ex. | break (%) | 5N (%) | (N/50 mm) | (N/50 mm) | 50-100% | 100-150% |
| 1 | 140.2 | 127.6 | 4.0 | 4.0 | 1.5 | −0.0 |
| 2 | 171.5 | 83.8 | 5.9 | 7.1 | 4.7 | 2.5 |
| 3 | 160.3 | 70.9 | 6.5 | 7.6 | 4.9 | 2.3 |
| 4 | 153.2 | 59.9 | 7.5 | 8.8 | 6.2 | 2.6 |
| 5 | 149.3 | 74.5 | 6.2 | 7.1 | 4.3 | 1.7 |
| 6 | 176 | 93.5 | 5.4 | 6.0 | 3.6 | 1.3 |
| 7 | 157.8 | 58.0 | 7.9 | 10.0 | 6.9 | 4.3 |

All values from Table 2 were determined following the WSP 100.4 test method.

The comparison of Examples 1 and 5 (both outside the invention) demonstrates that the CD elongation at 5N becomes higher (improves) when the sheet is underbonded as in Example 1. At the same time, CD elongation at break does not achieve the desired 150% in either case and is even slightly worse in Example 1. Also, the fact that the sum of slope 100-150% for Example 1 is negative proves that the sheet of Example 1 is no longer intact in elongation ranges exceeding 100%.

In contrast, the inventive examples achieve easy elongation of the nonwoven sheet and at the same time obtain high CD-elongations at max force more than 150% while keeping the nonwoven sheet material. Therefore, the meltblown layers formed from the thermoplastic elastomer functions as an adhesive keeping the nonwoven sheet intact while making it possible to underbond the spunbonded fibers without the nonwoven sheet delaminating during the elongation.

For example, the comparison of inventive Examples 2/6, instead of comparative Example 1, with comparative Example 5 demonstrates that the underbonded sheets have different and improved properties when the thermoplastic elastomer was used for the M layer. Not only does CD elongation at break become higher and exceed the desired 150% limit, also CD elongation at 5N becomes higher (improves) and the slope 100-150% remains positive.

The fact that both the slope 100-150% as well as the absolute tensile at 150% elongation of Example 2 is higher than that of Example 6 can be regarded as an indicator that the higher content of Vistamaxx (75 wt. %) in Example 2 keeps the nonwoven material intact better than in Example 6 (50 wt. % Vistamaxx).

### Examples 8-11

The examples as described in Table 3 were prepared on an SMMS (spunbonded, meltblown, meltblown, spunbonded) line as shown in FIG. 2, with the spunbonded layers being loft (L, comprising bicomponent fibers). Line parameters were a line speed of 290-305 m/min and a nip pressure of 56-66 N/mm. The bond pattern was an open dot bond pattern with a bonding area of 13.6%.

TABLE 3

| | | | | | | | | BW |
|---|---|---|---|---|---|---|---|---|
| | | coPP | PP/coPP | BW S1 | PP | coPP | PP/coPP | S2 |
| Ex. | PP S1 | S1 | ratio S1 | (g/m²) | S2 | S2 | ratio S2 | (g/m²) |
| 8 | 5.3 | 5.3 | 50/50 | 10.6 | 4.2 | 4.2 | 50/50 | 8.4 |
| 9 | 5.3 | 5.3 | 50/50 | 10.6 | 4.2 | 4.2 | 50/50 | 8.4 |
| 10 | 5.3 | 5.3 | 50/50 | 10.6 | 4.2 | 4.2 | 50/50 | 8.4 |
| 11 | 5.3 | 5.3 | 50/50 | 10.6 | 4.2 | 4.2 | 50/50 | 8.4 |

| | oTPE in | rem. | wt. % oTPE | BW MM | Calender Temp. | Calender Temp. |
|---|---|---|---|---|---|---|
| Ex. | MM | MM | in MM | (g/m²) | E-roll (° C.) | S-roll (° C.) |
| 8 | | 708FB | 0 | 3.4 | 99 | 91 |
| 9 | | 708FB | 0 | 3.4 | 147 | 131 |
| 10 | EXP520 | | 100 | 3.4 | 147 | 131 |
| 11 | EXP520 | | 100 | 3.4 | 99 | 91 |

The 708FB material is a standard polypropylene homopolymer meltblown grade from the company Borealis with an MFR of 800 g/10 min.

Examples 8, 9 and 10 are comparative examples outside the scope of the present invention, as they do not comprise a TPE material in the meltblown core layer (Examples 8, 9) and/or are not underbonded (Examples 9, 10). Example 11 is an inventive example. Measurement results for the nonwoven sheets of Examples 8-11 are shown in Table 4.

TABLE 4

| | CD tensile | CD tensile | % force of |
|---|---|---|---|
| | strength (TSCD) | elongation | TSCD at |
| | @break | @break | TE(100%) |
| Ex. | [N/50 mm] | [%] | [%] |
| 8 | 11.3 | 106.3 | 95.2 |
| 9 | 16.8 | 174.4 | 72 |
| 10 | 23.7 | 238.5 | 42.6 |
| 11 | 19.3 | 265.8 | 34.9 |

All values from Table 4 were determined following the WSP 100.4 test method.

As apparent, the examples that use TPE in the meltblown layer (Examples 10 and 11) have better tensile strength and much better elongation than the examples using standard meltblown grade materials (examples 8 and 9). Also, as apparent from the last column of Table 4, both examples that use TPE in the meltblown layer (Examples 10 and 11) show that tensile @100% is much less than 0.65 and even less than 0.55 of tensile @break. This comparison between the CD tensile force at 100% stretch to the CD tensile force at break is representative for a very easy elongation in the most useful range of below 100% stretch, in particular between 50-100% stretch.

Within the examples that use TPE in the meltblown layer (Examples 10 and 11), the underbonded inventive examples (Example 11) shows still better properties of elongation @break (beyond 250%) and a quotient of tensile @100% to tensile @break of less than 0.40.

Within the examples that use standard meltblown grade materials (examples 8 and 9), underbonding as in Example 8 makes these properties much worse instead of better.

To summarize, when utilizing an elastic polymer in the meltblown layer, it is possible to underbond the spunbonded loft fibers (calender temperature below usual bonding temperatures). Then the spunbonded fibers will not be the limiting factor for the stretchability and the elastic polymer in the meltblown layer functions as an adhesive layer keeping the nonwoven web intact. The elastic polymer does not only facilitate higher stretchability itself, but also allows the spunbonded fibers to be notably underbonded, while keeping the nonwoven web from delaminating, which is normally seen when underbonding SS and SMS nonwoven (SMMS).

1. A method for making a nonwoven sheet, the nonwoven sheet comprising two facing layers of spunbonded nonwoven material and a middle layer of meltblown nonwoven material, wherein the spunbonded material of the facing layers comprises crimped multicomponent fibers, the method comprising the following in-line steps:
spinning crimped multicomponent fibers and laying them onto a moving spinbelt to form a web preceding one of the facing layers;
meltblowing fibers and laying them onto the surface of the web formed in the previous step to form a web preceding the middle layer;
spinning crimped multicomponent fibers and laying them onto the surface of the web formed in the previous step to form a web preceding the other one of the facing layers; and
bonding the adjacent webs to form the nonwoven sheet by point-bond hot calendering; wherein
the fibers of the meltblown material of the middle layer comprise a thermoplastic elastomer (TPE); and
that the temperature of the engraved roll of the calender is at least 10° C. and/or 5% lower than the melting temperature, as determined with DSC according to ISO 11357-3, of the lowest melting component of the multicomponent fibers.

2. The method of claim 1, wherein the temperature of the engraved roll of the calender is at least 20° C. lower than the melting temperature of the lowest melting component of the crimped multicomponent fibers, and/or wherein the temperature of the engraved roll of the calender is at least 10% lower than the melting temperature of the lowest melting component of the crimped multicomponent fibers.

3. The method of claim 1, wherein the absolute temperature of the engraved roll of the calender is 130° C. or less.

4. The method of claim 1, wherein the temperature of the smooth roll of the calender is smaller than the temperature of the engraved roll of the calender.

5. The method of claim 1, wherein the nip pressure of the calender is between 20 and 100 N/mm; and/or wherein the line speed of the nonwoven sheet passing through the calender is between 100 and 400 n/min.

6. The method of claim 1, wherein the engraved roller is such that the number of bonding points per cm² of fabric surface is between 30 and 70, that the total area of the fabric surface taken up by the bonding points is between 8 and 15%, or both.

7. The method of claim 1, wherein one of the components of the crimped multicomponent fibers is a polypropylene (PP) material and another component of the crimped multicomponent fibers is either a different polypropylene (PP) material or a propylene-α-olefin copolymer (co-PP) material.

8. The method of claim 7, wherein the melting temperature of the polypropylene (PP) material, as determined with DSC according to ISO 11357-3, is between 15° and 170° C.; and/or wherein the melting temperature of the propylene-α-olefin copolymer (co-PP) material, as determined with DSC according to ISO 11357-3, is between 14° and 160° C.

9. The method of claim 1, wherein the melt flow rates of all polymer materials used as components in the multicomponent fibers is between 10 and 50 g/10 min, as determined according to ISO 1133 with conditions being 230° C. and 2.16 kg.

10. The method of claim 1, wherein at least 10 wt. % of the material forming for the meltblown fibers of the middle layer is a thermoplastic elastomer (TPE) material; and/or wherein the thermoplastic elastomer (TPE) material is a thermoplastic polyolefin elastomer (TPE-o) material.

11. A nonwoven sheet made by a method of claim 1, the nonwoven sheet comprising two facing layers of spunbonded nonwoven material and a middle layer of meltblown nonwoven material, wherein the spunbonded material of the facing layers comprises crimped multicomponent fibers, wherein
the meltblown material of the middle layer comprises a thermoplastic elastomer;
the nonwoven sheet is underbonded; and
the nonwoven sheet, in cross-machine direction (CD), has a tensile strength @100% elongation of less than 0.65 of its tensile strength @break, with tensile strength @100% elongation and tensile strength @break both being determined according to WSP 110.4.

12. The nonwoven sheet according to claim 11, the nonwoven sheet, in cross-machine direction (CD), has a tensile strength @100% elongation of less than 0.55 of its tensile strength @break, with tensile strength @100% elongation and tensile strength @break both being determined according to WSP 110.4.

13. The nonwoven sheet according to claim 11, wherein the nonwoven sheet has an extension at break in cross-machine direction (CD), when measured according to WSP 110.4, of at least 150%, a CD elongation at 5N, when measured according to WSP 110.4, of at least 75%, or both.

14. A laminate comprising the nonwoven sheet according to claim 11 and an elastically stretchable material.

15. A hygiene article comprising the nonwoven sheet according to claim 11, wherein the hygiene article is an open diaper having diaper ears and the nonwoven sheet or laminate forms at least part of the diaper ears.

16. The method of claim 1, wherein the temperature of the engraved roll of the calender is at least 40° C. lower than the melting temperature of the lowest melting component of the crimped multicomponent fibers, and/or wherein the temperature of the engraved roll of the calender is at least 30% lower than the melting temperature of the lowest melting component of the crimped multicomponent fibers.

17. The method of claim 1, wherein the absolute temperature of the engraved roll of the calender is 110° C. or less.

18. The method of claim 1, wherein the temperature of the smooth roll of the calender is smaller than the temperature of the engraved roll of the calender, wherein the temperature difference between the engraved and smooth rolls of the calender is less than 20° C.

19. The method of claim 1, wherein the nip pressure of the calender is between 30 and 70 N/mm; and/or wherein the line speed of the nonwoven sheet passing through the calender is between 200 and 350 m/min.

20. The method of claim 1, wherein one of the components of the crimped multicomponent fibers is a polypropylene (PP) material and another component of the crimped multicomponent fibers is a poly(propylene-ethylene) copolymer material.
